(19) United States
(12) Patent Application Publication
(10) Pub. No.: US 2020/0386702 A1
(43) Pub. Date: Dec. 10, 2020

(54) GAS SENSOR COMPRISING A HALIDE-BASED LAYER

(71) Applicant: Sumitomo Chemical Company Limited, Tokyo (JP)

(72) Inventors: Simon Godddard, Impington (GB); Nir Yaacobi-Gross, Cambrdgeshire (GB)

(73) Assignee: Sumitomo Chemical Company Limited, Tokyo (JP)

(21) Appl. No.: 16/767,337

(22) PCT Filed: Nov. 27, 2018
(86) PCT No.: PCT/GB2018/053419

§ 371 Date: May 27, 2020

(30) Foreign Application Priority Data
Nov. 29, 2017 (GB) 171985605

(51) Int. Cl. G01N 27/12; G01N 27/414; G01N 33/00; H01L 51/05

(52) U.S. Cl. CPC G01N 27/125; H01L 51/0562; G01N 33/0047; G01N 27/4141

(57) ABSTRACT

A gas sensor includes first and second electrodes (107, 109) and a first semiconductor layer (113) comprising a semiconducting transition halide or pseudohalide, for example copper thiocyanate, in electrical contact with the first and second electrodes. The semiconducting transition metal halide or pseudohalide provide that a semiconductor based gas sensor is sensitive to alkenes and can detect low concentrations of alkenes. Furthermore, the gas sensor may comprise a second semiconductor layer (111), different from the first semiconductor layer. The second semiconductor layer is preferably an organic semiconductor. The gas sensor may be a top-gate or bottom-gate thin film transistor (103: gate electrode; 105: gate dielectric) or a horizontal or vertical chemiresistor. The gas sensor may be used for detection of alkenes, for example ethylene or 1-MCP.

### FIELD

Embodiments of the present disclosure relate to semiconductor gas sensor devices and the use thereof in detection of gases. In some embodiments, the semiconductor gas sensor devices are configured for detection of alkenes.

### BACKGROUND

The use of thin film transistors as sensors is disclosed in, for example, Feng et al., Unencapsulated Air-stable Organic Field Effect Transistor by All Solution Processes for Low Power Vapor Sensing, SCIENTIFIC REPORTS 6:20671 DOI: 10.1038/srep20671 and Besar et al., Printable Ammonia Sensor Based on Organic Field Effect Transistor, ORGANIC ELECTRONICS, Volume 15, Issue 11, Pages 3221-3230 (November 2014).

Ethylene produced by plants can accelerate ripening of climacteric fruit, the opening of flowers, and the shedding of plant leaves. 1-methylcyclopropene (1-MCP) is known for use in inhibiting such processes.

Hirayama & Alonso, Ethylene Captures a Metal! Metal Ions are Involved in Ethylene Perception and Signal Transduction, PLANT CELL PHYSIOL 41(5) 548 (2000) discloses evidence for the role of copper in ethylene perception.

United States Patent Pub. No. 2013/0273665 discloses a sensor device including a transition metal complex capable of interacting with a carbon-carbon multiple bond moiety.

Han et al., Achievement of High-response Organic Field-effect Transistor NO2 Sensor by Using the Synergistic Effect of ZnO/PMMA Hybrid Dielectric and CuPc/Pentacene Heterojunction, SENSORS 16 1763 (2016) discloses OFET-based NO₂ sensors using a synergistic effect of a zinc oxide/poly(methyl methacrylate) (ZnO/PMMA) hybrid dielectric and CuPc/Pentacene heterojunction.

### SUMMARY

The present inventors have found that a semiconducting transition metal halide or pseudohalide may be used to enhance detection of gases, as compared to devices where such a compound is not present. The gas sensor is capable of detecting gases at low concentrations.

In some embodiments of the present disclosure a gas sensor comprising a semiconducting transition metal halide or a pseudohalide is provided for detecting alkenes. In such embodiments, the gas detector is capable of detecting the alkenes at low concentrations.

Accordingly, in a first aspect, according to some embodiments of the present disclosure, a gas sensor includes first and second electrodes and a first semiconducting layer comprising a semiconducting transition metal halide or pseudohalide in electrical contact with the first and second electrodes.

In a second aspect, according to some embodiments of the present disclosure, the disclosure provides a method of identifying the presence and/or concentration of at least one target gas in an environment, the method comprising measurement of a response of the gas sensor according to the first aspect in the environment and determining from the measured parameter if the at least one target gas is present and/or determining a concentration of the at least one target gas.

### DESCRIPTION OF THE DRAWINGS

The disclosure will now be described in detail with reference to the Figures in which:

FIG. 1 illustrates a bottom gate, bottom contact organic thin film transistor for use as a gas sensor according to an embodiment;

FIG. 2 illustrates a bottom gate, bottom contact organic thin film transistor for use as a gas sensor according to an embodiment;

FIG. 3 illustrates a bottom gate, top contact organic thin film transistor for use as a gas sensor according to an embodiment;

FIG. 4 illustrates a bottom gate, top contact organic thin film transistor for use as a gas sensor according to an embodiment;

FIG. 5 illustrates a top gate organic thin film transistor for use as a gas sensor according to an embodiment;

FIG. 6 illustrates a top gate organic thin film transistor for use as a gas sensor according to an embodiment;

FIG. 7 illustrates a top gate organic thin film transistor for use as a gas sensor according to an embodiment;

FIG. 8 illustrates a bottom contact horizontal chemiresistor for use as a gas sensor according to embodiments of the disclosure;

FIG. 9 illustrates a top contact horizontal chemiresistor for use as a gas sensor according to embodiments of the disclosure;

FIG. 10 illustrates a vertical chemiresistor for use as a gas sensor according to an embodiment;

FIG. 11 illustrates a vertical chemiresistor for use as a gas sensor according to an embodiment;

FIG. 12A is a graph of drain current vs. time of a bottom-contact OTFT according to an embodiment upon exposure to ethylene;

FIG. 12B shows the change of FIG. 12A as a percentage change from a baseline current in a nitrogen-only atmosphere;

FIG. 13 is a comparison of response to ethylene of bottom-contact OTFTs according to an embodiment compared to a comparative bottom-contact OTFT;

FIG. 14A is a graph of drain current vs. time of a bottom gate, top-contact OTFT according to an embodiment upon exposure to 1-MCP;

FIG. 14B shows the change of FIG. 14A as a percentage change from a baseline current in a nitrogen-only atmosphere;

FIG. 15 is a comparison of response to 1-MCP of bottom gate, top-contact OTFTs according to an embodiment compared to a comparative bottom gate, top-contact OTFT;

FIG. 16 shows a change in the drain current vs. time as a percentage change from a baseline current in a nitrogen-only atmosphere according to an embodiment and a comparative bottom gate, bottom-contact OTFT;

FIG. 17 is a comparison of response to butyl acetate at different concentrations of bottom gate, bottom-contact OTFTs according to an embodiment compared to a comparative bottom-contact OTFT; and

FIG. 18 is a transfer scan showing the drain current according to an embodiment compared to a comparative bottom gate, bottom-contact OTFT.

### DETAILED DESCRIPTION

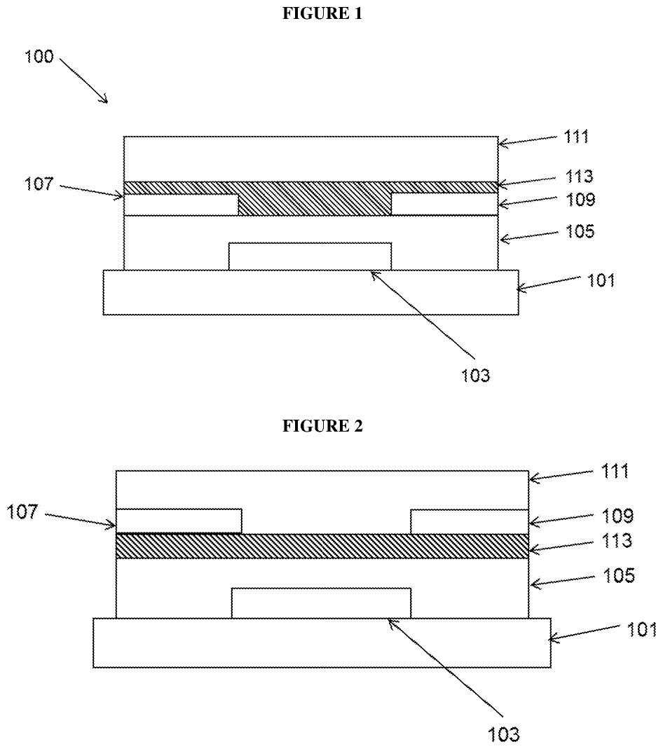

FIG. 1, which is not drawn to any scale, is a schematic illustration of a bottom contact, bottom gate TFT (BG-TFT) suitable for use as a gas sensor. The bottom contact BG-TFT comprises a gate electrode 103 supported on a substrate 101; source and drain electrodes 107, 109; a dielectric layer 105 between the gate electrode and the source and drain electrodes; a first semiconducting layer 113 comprising a semiconducting transition metal halide or pseudohalide extending between the source and drain electrodes 107, 109; and a second semiconducting layer 111 in contact with the first semiconducting layer 113. The first semiconducting layer 113 may at least partially or completely cover the source and drain electrodes.

The second semiconducting layer is different from the first semiconducting layer. Preferably, the second semiconducting layer is an organic semiconducting (OSC) layer. Preferably, the second semiconducting layer does not comprise a semiconducting transition metal halide or pseudohalide.

As used herein, by a material “over” a layer is meant that the material is in direct contact with the layer or is spaced apart therefrom by one or more intervening layers.

As used herein, by a material “on” a layer is meant that the material is in direct contact with that layer.

A layer “between” two other layers as described herein may be in direct contact with each of the two layers it is between or may be spaced apart from one or both of the two other layers by one or more intervening layers. Thus, in some embodiments, the layer “between” two other layers as described herein may be considered to be an “interlayer”.

FIG. 2, which is not drawn to any scale, is a schematic illustration of a bottom contact BG-TFT as described with respect to FIG. 1 except that the first semiconducting layer 113 is between the dielectric layer 105 and the source and drain electrodes 107, 109. The first semiconducting layer 113 contacts the second semiconducting layer 111 in the region between the source and drain electrodes.

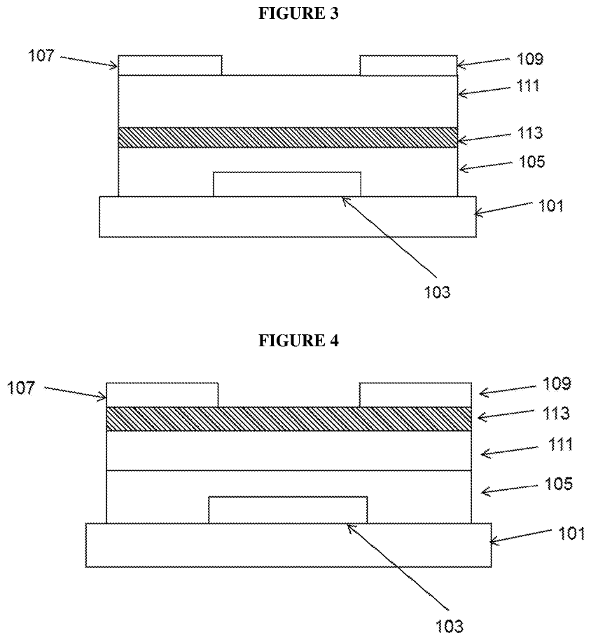

FIG. 3 is a schematic illustration of a top-contact BG-TFT. The top-contact BG-TFT is as described with reference to FIG. 1 except that the second semiconducting layer 111 is between the dielectric layer 105 and the source and drain electrodes 107, 109 and the source and drain electrodes 107, 109 and the first semiconducting layer 113 are separated by the organic semiconducting layer 111.

FIG. 4 is a schematic illustration of a top-contact BG-TFT as described with reference to FIG. 3 except that the first semiconducting layer 113 is between the source and drain electrodes 107, 109 and the organic semiconducting layer 111.

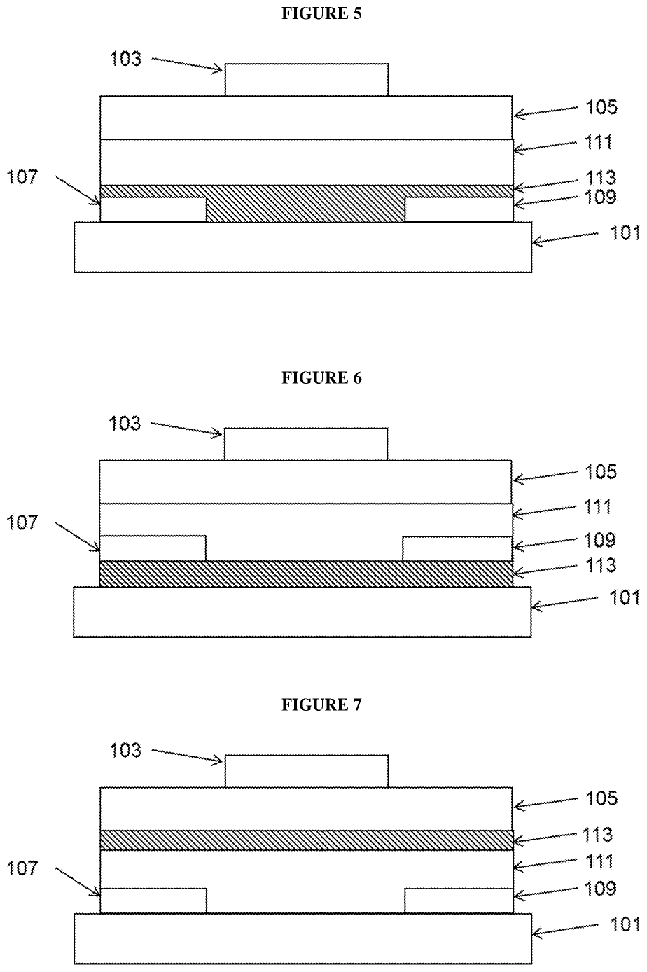

FIG. 5 is a schematic illustration of a top gate TFT suitable for use as a gas sensor. The top gate TFT comprises source and drain electrodes 107, 109 supported on a substrate; an second semiconducting layer 111; a gate electrode 103; a dielectric layer 105 between the gate electrode 103 and the second semiconducting layer 111; and a first semiconducting layer comprising a semiconducting transition metal halide or pseudohalide 113 between the source and drain electrodes 107, 109 and the second semiconducting layer 111 and in contact with the second semiconducting layer 111. The dielectric layer of the top-gate TFT is a gas-permeable material, preferably an organic material, which allows permeation of the gas or gases to be sensed through the dielectric layer. FIG. 5 illustrates a top-gate TFT in which all of a surface of an underlying layer is covered by the dielectric layer. In other embodiments, at least some of the underlying layer is not covered by the dielectric layer.

FIG. 6 is a schematic illustration of a top gate TFT suitable for use as a gas sensor as described in FIG. 5 except that the first semiconducting layer 113 is between the substrate and the source and drain electrodes 107, 109.

FIG. 7 is a schematic illustration of a top gate TFT suitable for use as a gas sensor as described in FIG. 5 except that the first semiconducting layer 113 is between the dielectric layer 105 and the organic semiconducting layer 111.

TFT gas sensors as described herein are preferably BG-TFTs. In the case of a BG-TFT the first semiconducting layer is preferably between and in contact with a dielectric layer and the second semiconducting layer or between and in contact with source and drain electrodes and the second semiconducting layer.

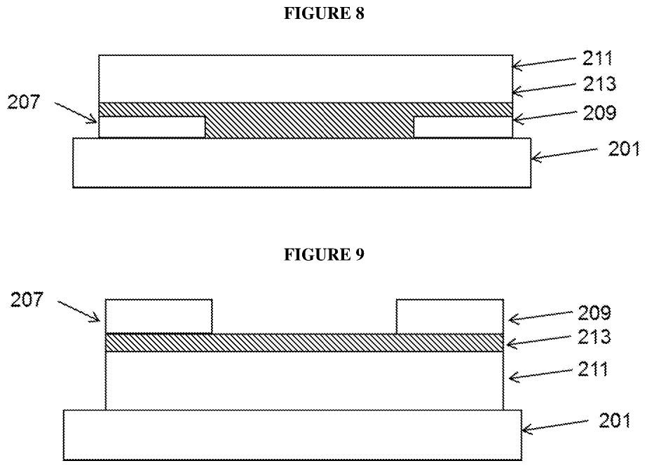

FIG. 8 illustrates a bottom contact horizontal chemiresistor according to an embodiment suitable for use as a sensor as described herein. By “bottom contact chemiresistor” as used herein is meant that electrodes of the chemiresistor lie between a substrate and a semiconducting layer of the chemiresistor

The chemiresistor comprises first and second electrodes 207 and 209 supported on a substrate and having a first semiconducting layer comprising a semiconducting transition metal halide or pseudohalide 213 formed thereon. A second semiconducting layer 211 is provided between, and in electrical connection with, the first and second electrodes. The first and second electrodes may be interdigitated. The chemiresistor may be supported on any suitable substrate 201, for example a glass or plastic substrate.

In another embodiment (not shown), the gas sensor is a bottom contact horizontal chemiresistor as described with reference to FIG. 8 except that the first semiconducting layer is formed on the second semiconducting layer such that the second semiconducting layer lies between the first semiconducting layer and the first and second electrodes.

FIG. 9 illustrates a top contact horizontal chemiresistor according to an embodiment suitable for use as a sensor as described herein. By “top contact chemiresistor” as used herein is meant that a semiconducting layer of the chemiresistor lies between electrodes and a substrate of the chemiresistor.

Integers of the chemiresistor of FIG. 9 are as described with reference to FIG. 8. The first semiconducting layer 213 lies between the second semiconducting layer and the electrodes.

In another embodiment (not shown), the gas sensor is a top contact horizontal chemiresistor as described with reference to FIG. 9 except that the first semiconducting layer is between the substrate and the second semiconducting layer.

The first and second electrodes of a horizontal chemiresistor as described herein may be separated by a distance of between 5-500 microns, optionally 50-500 microns.

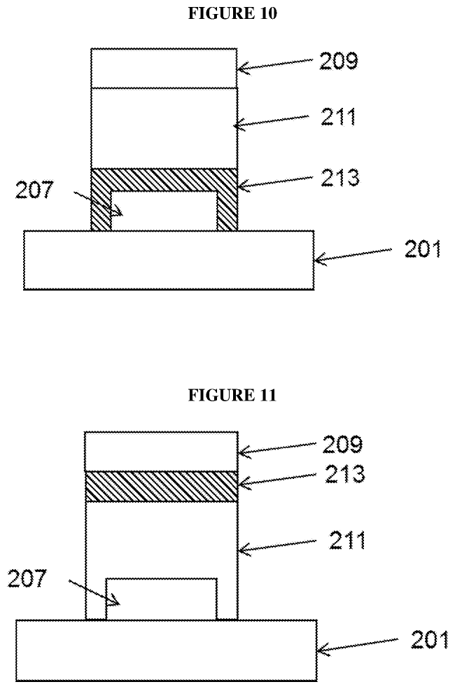

FIG. 10 illustrates a vertical chemiresistor according to an embodiment suitable for use as a gas sensor as described herein. The chemiresistor comprises a first, bottom electrode 207 having a first semiconducting layer 213 formed thereon; a second, top electrode 209 over the first electrode; and a second semiconducting layer 211 between, and in electrical connection with, the first and second electrodes. The bottom electrode 207 lies between the substrate and both the second semiconducting layer 211 and the second, top electrode 209.

FIG. 11 illustrates another vertical chemiresistor according to an embodiment suitable for use as a sensor as described herein. Integers of the chemiresistor of FIG. 11 are as described with reference to FIG. 10. The first semiconducting layer 213 lies between the second semiconducting layer and the second, top electrode 209.

In a further embodiment (not shown), a vertical chemiresistor comprises two first semiconducting layers spaced apart by a second semiconducting layer.

The first and second electrodes of a vertical chemiresistor as described herein may be separated by a distance of between 20 nm-10 microns, optionally 50-500 nm.

The gas sensors have been described herein with reference to sensors comprising or consisting of a first semiconducting layer of a transition metal halide or pseudohalide and a separate second semiconducting layer. In other embodiments, the second semiconducting layer is not present, the first semiconducting layer being the only semiconducting layer of the gas sensor. Optionally according to these embodiments, the layer of transition metal halide or pseudohalide directly contacts the first and second electrodes.

In use, a gas sensor as described herein is exposed to a gaseous atmosphere and is connected to apparatus for measuring a response of the gas sensor to the atmosphere resulting from absorption of one or more gases in the atmosphere. In the case of a chemiresistor the response may be a change in resistance of the chemiresistor. In the case of a TFT the response may be a change in the drain current.

The gas sensor is preferably for sensing an alkene, more preferably 1-methylcyclopropene (1-MCP) and/or ethylene. In use, the gas sensor may be placed in an environment in which alkenes may be present in the environmental atmosphere, for example a warehouse in which harvested climacteric fruits and/or cut flowers are stored and in which ethylene may be generated.

The presence and/or concentration of ethylene may be determined using the gas sensor. If ethylene concentration reaches or exceeds a predetermined threshold value, which may be any value greater than 0, then 1-MCP may be released from a 1-MCP source to retard the effect of the ethylene, such as ripening of fruit or opening of flowers in the environment.

Optionally, 1-MCP may be released into the atmosphere if 1-MCP concentration falls to or below a threshold 1-MCP concentration value as determined by the gas sensor.

1-MCP may be released automatically from a 1-MCP source or an alert or instruction may be generated to manually release 1-MCP from a 1-MCP source in response to signal from the gas sensor upon determination that 1-MCP concentration is at or below a threshold that is a positive value and/or in response to a determination that ethylene concentration is at or exceeds a threshold which may be 0 or a positive value.

In an embodiment, the gas sensor can be used for sensing an ester, more preferably butyl acetate (n-butyl acetate). In use, the gas sensor may be placed in an environment in which esters may be present in the environmental atmosphere, for example a warehouse in which harvested apples (which naturally emit butyl acetate) are stored.

The gas sensor may be in wired or wireless communication with a controller which controls automatic release of 1-MCP from a 1-MCP source and/or a user interface providing information on the presence and/or concentration of ethylene and/or 1-MCP in the environment.

An environment in which an alkene may be present may be divided into a plurality of regions if the concentration of an alkene or alkenes may differ between regions, each region comprising a gas sensor according to the present disclosure and a source of 1-MCP. For example, a warehouse may comprise a plurality of regions.

The gas sensor may comprise one or more control gas sensors, optionally one or more TFT gas sensors, to provide a baseline for measurements take into account variables such as one or more of humidity, temperature, pressure, variation of sensor parameter measurements over time (such as variation of TFT sensor drain current over time), and gases other than a target gas or target gases in the atmosphere. One or more control gas sensors may be isolated from the atmosphere, for example by encapsulation of the or each control sensor, to provide a baseline measurement other than gases in the atmosphere.

The response of a gas sensor as described herein to background gases other than the target gases for detection, for example air or water vapour, may be measured prior to use to allow subtraction of the background from measurements of the gas sensor when in use.

Transition Metal Halide or Pseudohalide

The first semiconducting layer may consist of the transition metal halide or pseudohalide or may comprise one or more further materials. Preferably, the first semiconducting layer consists of the transition metal halide or pseudohalide.

The first semiconducting layer may have a thickness of 1-20 nm, optionally 2-10 nm when used in combination with a second semiconducting layer. The first semiconducting layer may have a thickness of 10-100 nm, optionally 40-60 nm when used without a separate semiconducting layer, optionally when the transition metal halide or pseudohalide is the only layer between the first and second electrodes.

The transition metal halide or pseudohalide may be a metal complex, optionally a coordination polymer. The transition metal of the transition metal halide or pseudohalide is optionally copper (I), silver (I) or cobalt and is preferably Cu (I).

Optionally, the halide of a semiconducting transition metal halide is selected from fluoride, chloride, bromide, iodide or astatide.

Optionally, the pseudohalide of a semiconducting transition metal pseudohalide is selected from thiocyanate, selenocyanate and tellurocyanate.

Preferably, the transition metal halide or pseudohalide is selected from copper thiocyanate (CuSCN); silver thiocyanate (AgSCN); cuprous iodide (CuI). copper selenocyanate (CuSeCN) and copper tellurocyanate (CuTeCN). Copper thiocyanate is particularly preferred.

Preferably, the first semiconducting layer is deposited from a formulation comprising the semiconducting transition metal halide or pseudohalide dissolved or dispersed in one or more solvents. Solvents for CuSCN include, without limitation, dialkylsulfides, for example diethylsulfide and dipropylsulfide; and aqueous ammonium hydroxide.

Preferably, the first and second semiconducting layers are formed by depositing formulations comprising a semiconducting material and the transition metal halide or pseudohalide respectively dissolved or dispersed in one or more solvents. This is particularly preferred if the second semiconducting layer comprises or consists of one or more organic semiconductors. The second semiconducting layer may be deposited onto the layer comprising the transition metal halide or pseudohalide or vice-versa.

The solvents of the formulations for depositing the first and second semiconducting layers may be selected such that the first of these two layers to be deposited does not dissolve when the other layer is deposited onto it.

Deposition techniques for depositing the first and second semiconducting layers include coating and printing methods, for example spin coating dip-coating, slot-die coating, ink jet printing, gravure printing, flexographic printing and screen printing.

Electrodes

The first and second electrodes of the gas sensor are preferably source and drain electrodes of first and second TFTs, or first and second electrodes of first and second chemiresistors.

The first and second electrodes can be selected from a wide range of conducting materials for example a metal (e.g. gold), metal alloy, metal compound (e.g. indium tin oxide) or conductive polymer.

In the case of a TFT, the gate electrode may be selected from any conducting material, for example a metal (e.g. aluminium), a metal alloy, or a conductive metal compound (e.g. a conductive metal oxide such as indium tin oxide).

The length of the channel defined between the source and drain electrodes of the first and second source and drain and gate electrodes of the first and second TFTs may be up to 500 microns, but preferably the length is less than 200 microns, more preferably less than 100 microns.

Second Semiconductor Layer

The second semiconductor layer may comprise or consist of one or more organic semiconductors or one or more inorganic semiconductors other than a semiconducting transition metal halide or pseudohalide.

Organic semiconductors as described herein may be selected from conjugated non-polymeric semiconductors; polymers comprising conjugated groups in a main chain or in a side group thereof; and carbon semiconductors such as graphene and carbon nanotubes.

An organic second semiconductor layer may comprise or consist of a semiconducting polymer and/or a non-polymeric organic semiconductor. The organic semiconductor layer may comprise a blend of a non-polymeric organic semiconductor and a polymer.

Exemplary organic semiconductors are disclosed in WO 2016/001095, the contents of which are incorporated herein by reference.

An organic second semiconductor layer of a BG-OTFT preferably comprises or consists of only one organic semiconductor. An organic second semiconductor layer of top-gate organic thin film transistors is preferably a mixture of a non-polymeric and polymeric organic semiconductor.

The organic semiconducting layer may be deposited by any suitable technique, including evaporation and deposition from a solution comprising or consisting of one or more organic semiconducting materials and at least one solvent. Exemplary solvents include benzenes with one or more alkyl substituents, preferably one or more C₁₋₁₀ alkyl substituents, such as toluene, xylene and trimethylbenzene; tetralin; and chloroform.

Optionally, the organic semiconducting layer of an organic thin film transistor has a thickness in the range of about 10-200 nm.

Exemplary inorganic semiconductors include, without limitation, n-doped silicon; p-doped silicon; compound semiconductors, for example III-V semiconductors such as GaAs or InGaAs; or doped or undoped metal oxides.

Dielectric Layer

The dielectric layer of TFTs described herein comprises a dielectric material. Preferably, the dielectric constant, k, of the dielectric material is at least 2 or at least 3. The dielectric material may be organic, inorganic or a mixture thereof. Preferred inorganic materials include SiO₂, SiNx and spin-on-glass (SOG). Preferred organic materials are polymers and include insulating polymers such as poly vinylalcohol (PVA), polyvinylpyrrolidine (PVP), acrylates such as polymethylmethacrylate (PMMA) and benzocyclobutanes (BCBs), poly(vinyl phenol) (PVPh), poly(vinyl cinnamate) P(VCn), poly(vinylidene fluoride-co-hexafluoropropylene) P(VDF-HFP), P(VDF-TrFE-CTFE), and self-assembled monolayers, e.g. silanes, on oxide. The polymer may be crosslinkable. The insulating layer may be formed from a blend of materials or comprise a multi-layered structure. In the case of a bottom-gate device, the gate electrode may be reacted, for example oxidised, to form a dielectric material.

The dielectric material may be deposited by thermal evaporation, vacuum processing or lamination techniques as are known in the art. Alternatively, the dielectric material may be deposited from solution using, for example, spin coating or ink jet printing techniques and other solution deposition techniques discussed above. In the case of a bottom gate TFT, the dielectric material should not be dissolved if the first or second semiconducting layer is deposited onto it from a solution or dispersion. In the case of a top-gate TFT, the first or second semiconducting layer should not be dissolved if the dielectric is deposited onto it from solution.

Techniques to avoid such dissolution include: use of orthogonal solvents for example use of a solvent for deposition of second semiconducting layer which does not dissolve the dielectric layer in the case of a bottom gate device or vice versa in the case of a top gate device; cross linking of the dielectric layer before deposition of the first or second semiconducting layer in the case of a bottom gate device; or deposition from solution of a blend of the dielectric material and an organic semiconductor followed by vertical phase separation as disclosed in, for example, L. Qiu, et al., Adv. Mater. 2008, 20, 1141.

The thickness of the dielectric layer is preferably less than 2 micrometres, more preferably less than 500 nm.

The substrate of a sensor as described herein may be any insulating substrate, optionally glass or plastic.

Use of a gas sensor has been described herein with reference to 1-MCP and ethylene, however it will be appreciated that it may be applied to other alkenes, for example any alkene which is gaseous at 20° C. and 1 atm, e.g. a C₁₋₅ alkene.

A gas sensor as described herein may be used in sensing thiols.

### EXAMPLES

### Device Example 1

On a PEN substrate carrying an aluminium gate electrode was formed a crosslinked dielectric layer by spin-coating and crosslinking an insulating polymer to a thickness of 60-300 nm. Gold source and drain electrodes were formed on the dielectric layer by thermal evaporation. Copper thiocyanate was deposited onto the source and drain electrodes by spin-coating from diethylsulfide to form a layer of 5 nm thickness. Semiconducting Polymer 1, illustrated below, was formed over the dielectric layer and source and drain electrodes by spin-coating from 1,2,4-trimethylbenzene solution to a thickness of 40 nm to form bottom contact Device Example 1.

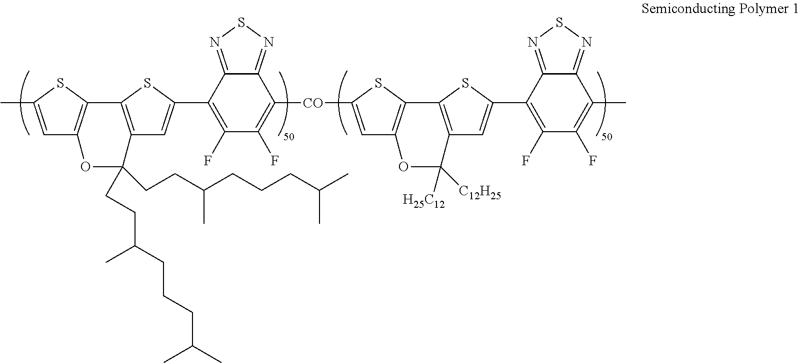

### Comparative Device 1

A device was prepared as described in Device Example 1 except that the layer of copper thiocyanate was not formed and the organic semiconductor layer was deposited directly onto the source and drain electrodes.

### Device Example 2

A device was prepared as described for Device Example 1 except that the copper thiocyanate layer was formed directly on the insulating layer; the organic semiconducting layer was formed on the copper thiocyanate layer; and the source and drain electrodes were formed on the organic semiconducting layer to form a top-contact device.

### Comparative Device 2

A device was prepared as described in Device Example 2 except that the layer of copper thiocyanate was not formed and the source and drain electrodes were deposited directly onto the organic semiconductor layer.

### Comparative Device 3

A device was prepared as described for Device Example 1, except that Semiconducting Polymer 1 was not formed over the dielectric layer and source and drain electrodes.

Ethylene Response

The responses of Device Example 1 and Comparative Device 1 upon exposure to ethylene gas were measured by monitoring the level of the drain current as a function of time.

The OTFT was driven at a constant finite voltage of Vg=Vds=−4V under dry nitrogen (100 cc/min) before introducing ethylene at a concentration of between 10 to 500 ppm in nitrogen at the same flow rate for 1 hour. After 1 hour, ethylene was removed from the nitrogen gas flow. Measurements were conducted at 20° C.

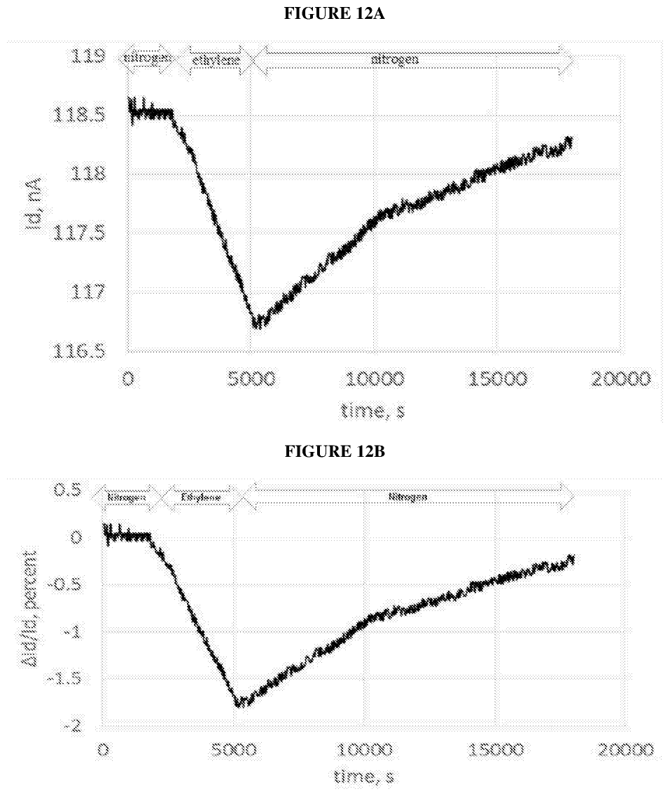

FIG. 12A shows a sharp fall in drain current upon exposure to ethylene with a recovery upon return to a nitrogen-only atmosphere. FIG. 12B shows this change as a percentage change from a baseline current in a nitrogen-only atmosphere.

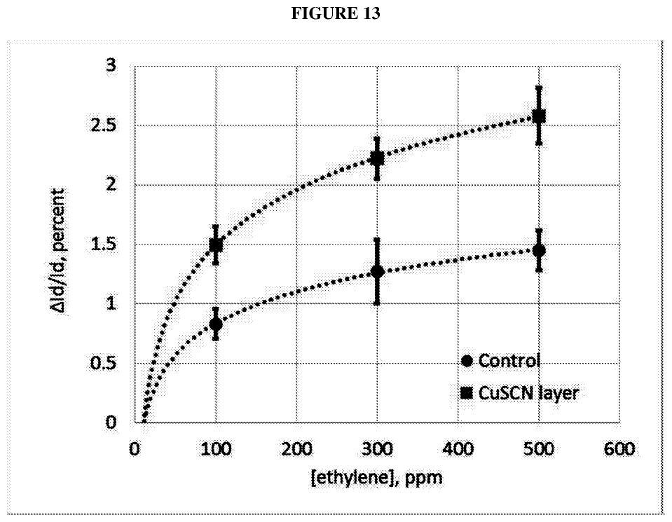

FIG. 13 shows a significantly stronger response of Device Example 1 to ethylene as compared to Comparative Device 1. The responses shown in this figure for each of Device Example 1 and Comparative Device 1 are an average for 3 devices. Error bars represent the standard deviation.

1-MCP Response

An alpha-cyclodextrin matrix containing 1-MCP (4.3 wt %) was added to water to displace the 1-MCP into a bottle purged with nitrogen (50 cc/min). The nitrogen gas carried the 1-MCP through a gas tight container containing Device Example 2.

Device Example 2 was biased (Vg=Vds=−4V) under high relative humidity nitrogen (50 cc/min) before introducing 1-MCP (50 ppb to 3000 ppb) in nitrogen at the same flow rate for 30 minutes. After 30 minutes the 1-MCP gas was stopped and the flow was returned to nitrogen.

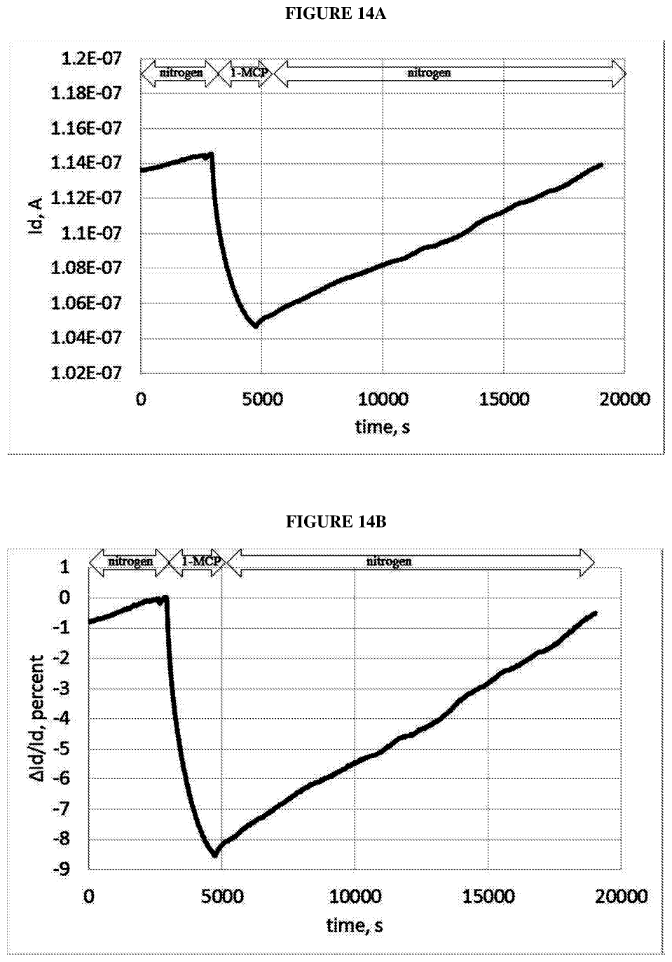

FIG. 14A shows a sharp fall in drain current upon exposure to 1-MCP with a recovery upon return to a nitrogen-only atmosphere. FIG. 14B shows this change as a percentage change from a baseline current in a nitrogen-only atmosphere.

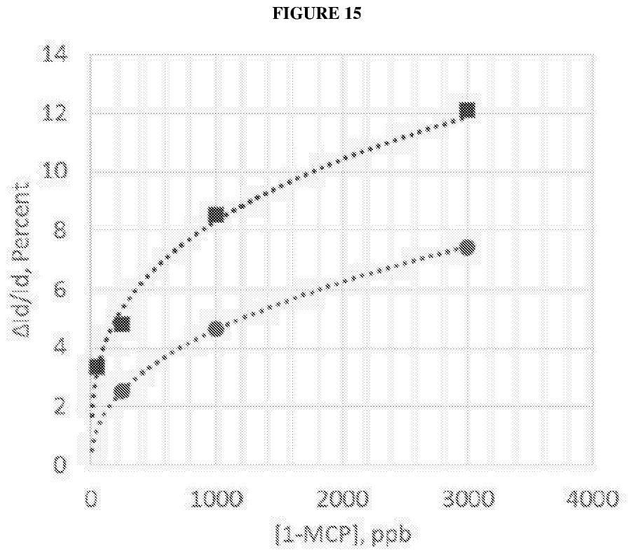

FIG. 15 shows a significantly stronger response of Device Example 2 to 1-MCP as compared to Comparative Device 2.

Butyl Acetate Response

The responses of Device Example 1 and Comparative Device 1 upon exposure to butyl acetate (2000 ppm) were measured by monitoring the drain current as a function of time.

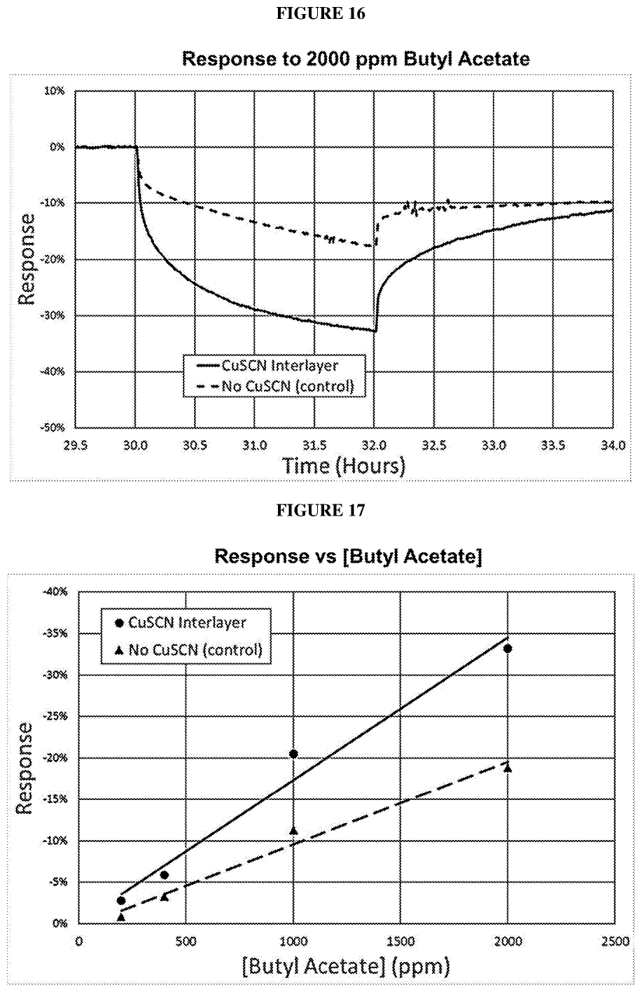

FIG. 16 shows a sharp fall in drain current upon exposure to butyl acetate with a recovery upon return to a nitrogen-only atmosphere.

The response of Device Example 1 and Comparative Device 1 upon exposure to butyl acetate at concentrations between 200 ppm and 2000 ppm was measured.

FIG. 17 shows that Device Example 1 was more sensitive to butyl acetate at all concentrations as compared to Comparative Device 1.

Removal of Organic Semiconductor (OSC) Layer

The drain current of Device Example 1 and Comparative Device 3 was measured immediately after fabrication of these devices.

The OTFT was biased (Vds=−4 V and Vg scanned from +0.5 V to −4 V) in air at 20° C.

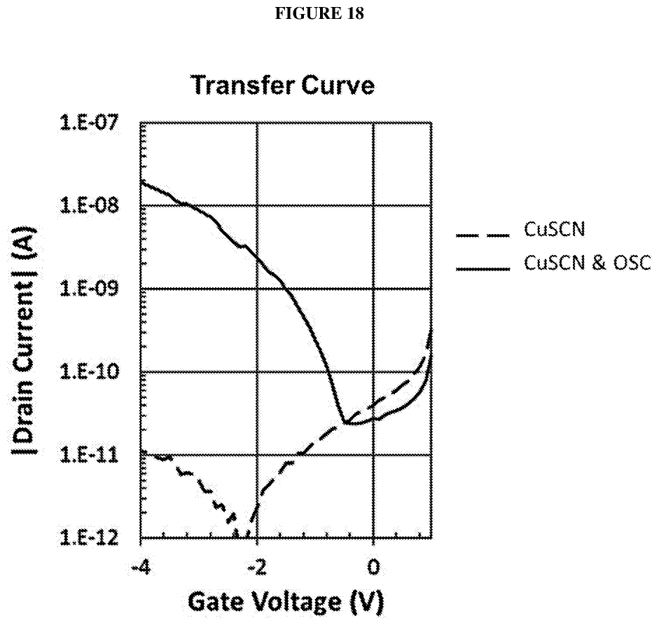

FIG. 18 is a transfer scan showing that that the drain current of Device Example 1 was higher than for Comparative Device 3. FIG. 18 illustrates that, in the absence of an OSC (as in Comparative Device 3), the drain current is negligible. Without wishing to be bound by theory, it is thought that the observed negligible drain current for Comparative Device 3 was exclusively due to a leakage current between the gate and drain electrodes.

Although the present disclosure has been described in terms of specific exemplary embodiments, it will be appreciated that various modifications, alterations and/or combinations of features disclosed herein will be apparent to those skilled in the art without departing from the scope of the disclosure as set forth in the following claims.

1. A gas sensor, comprising:
first and second electrodes; and
a first semiconducting layer comprising a semiconducting transition metal halide or pseudohalide in electrical contact with the first and second electrodes.

2. A gas sensor according to claim 1, further comprising:
a second semiconductor layer which is different from, and in contact with, the first semiconducting layer.

3. A gas sensor according to claim 2, wherein the second semiconductor layer comprises an organic semiconductor.

4. A gas sensor according to claim 1, wherein the sensor comprises a thin film transistor in which the first and second electrodes are source and drain electrodes of the thin film transistor, and wherein the thin film transistor further comprising a gate electrode and a dielectric layer.

5. A gas sensor according to claim 4, wherein the thin film transistor comprises a bottom gate thin film transistor (BG-TFT).

6. A gas sensor according to claim 5, wherein the BG-TFT comprises a bottom contact TFT.

7. A gas sensor according to claim 5, wherein the BG-TFT comprises a top contact TFT.

8. A gas sensor according to claim 4, wherein the thin film transistor comprises a top gate thin film transistor.

9. A gas sensor according to claim 2, wherein the first semiconducting layer is between the dielectric layer and the second semiconducting layer.

10. A gas sensor according to claim 2, wherein the first semiconducting layer is between the second semiconducting layer and the source and drain electrodes.

11. A gas sensor according to claim 1 wherein the gas sensor comprises a chemiresistor.

12. A gas sensor according to claim 11, wherein the gas sensor comprises a vertical chemiresistor.

13. A gas sensor according to claim 1, wherein the transition metal halide or pseudohalide comprises a coordination polymer.

14. A gas sensor according to claim 13, wherein the coordination polymer comprises copper thiocyanate.

15. A method for identifying the presence and/or concentration of a target gas in an environment, comprising:
measuring a response of the gas sensor according to claim 1 in the environment; and
determining from the measured response a presence and/or a concentration of the target gas in the environment.

16. A method according to claim 15, wherein the target gas is an alkene or an ester.

17. A method according to claim 16, wherein the alkene is ethylene, 1-methylcyclopropene and/or butyl acetate.

18. A method of forming a gas sensor according to claim 1, the method comprising the steps of depositing the first semiconducting layer from a solution or suspension.
